(19) United States
(12) Patent Application Publication
(10) Pub. No.: US 2026/0198870 A1
(43) Pub. Date: Jul. 16, 2026

(54) X-RAY CT APPARATUS

(71) Applicants: KEIO UNIVERSITY, Tokyo (JP); Canon Kabushiki Kaisha, Tokyo (JP)

(72) Inventors: Yoshitake YAMADA, Tokyo (JP); Masahiro JINZAKI, Tokyo (JP); Minoru YAMADA, Hino (JP); Yoichi YOKOYAMA, Tokyo (JP); Ryotaro TANAKA, Nasushiobara (JP)

(73) Assignees: KEIO UNIVERSITY, Tokyo (JP); Canon Kabushiki Kaisha, Tokyo (JP)

(21) Appl. No.: 19/442,144

(22) Filed: Jan. 7, 2026

(30) Foreign Application Priority Data
Jan. 10, 2025 (JP) 2025-004194

(51) Int. Cl. A61B 6/03; A61B 6/00; A61B 6/04

(52) U.S. Cl. CPC A61B 6/035; A61B 6/0407; A61B 6/4435

(57) ABSTRACT

According to one embodiment, an X-ray CT apparatus includes a scanner and a stand. The scanner includes a rotating unit configured to rotatably hold an imaging mechanism around a center axis of an opening, and a fixing unit configured to rotatably hold the rotating unit around the center axis, the fixing unit having a symmetric shape with respect to a horizontal axis perpendicular to the center axis. The stand is configured to support a side surface of the scanner, the stand having a symmetric shape with respect to a vertical axis perpendicular to the horizontal axis.

### CROSS-REFERENCE TO RELATED APPLICATION

This application is based upon and claims the benefit of priority from prior Japanese Patent Application No. 2025-004194, filed January 10, 2025, the entire contents of which are incorporated herein by reference.

### FIELD

Embodiments described herein relate generally to an X-ray CT apparatus.

### BACKGROUND

An X-ray CT (Computed Tomography) apparatus is an apparatus that executes X-ray CT imaging by a scanner, and there is known an X-ray CT apparatus including a stand that supports the scanner from one side surface of the scanner (hereinafter referred to as "cantilever X-ray CT apparatus"). The cantilever X-ray CT apparatus is classified into a structure in which the stand supports a right side surface of the scanner, as viewed from the side on which a bed is installed (this structure is referred to as "right-hand cantilever structure"), and a structure in which the stand supports a left side surface of the scanner (this structure is referred to as "left-hand cantilever structure").

### BRIEF DESCRIPTION OF THE DRAWINGS

FIG. 1 is a diagram illustrating a configuration of an X-ray CT apparatus according to an embodiment.

FIG. 2 is a perspective view illustrating a configuration of a scanner, a stand and a bed.

FIG. 3 is a perspective view illustrating a detailed configuration of the scanner and the stand.

FIG. 4 is a perspective view illustrating a right-hand cantilever structure and a left-hand cantilever structure.

FIG. 5 is a flowchart illustrating an operation of the X-ray CT apparatus according to the embodiment.

### DETAILED DESCRIPTION

In general, according to one embodiment, an X-ray CT apparatus includes a scanner and a stand. The scanner includes a rotating unit configured to rotatably hold an imaging mechanism around a center axis of an opening, and a fixing unit configured to rotatably hold the rotating unit around the center axis, the fixing unit having a symmetric shape with respect to a horizontal axis perpendicular to the center axis. The stand is configured to support a side surface of the scanner, the stand having a symmetric shape with respect to a vertical axis perpendicular to the horizontal axis.

Hereinafter, an embodiment is described with reference to the accompanying drawings. Parts denoted by identical reference signs are regarded as identical parts, and an overlapping description is omitted unless where necessary.

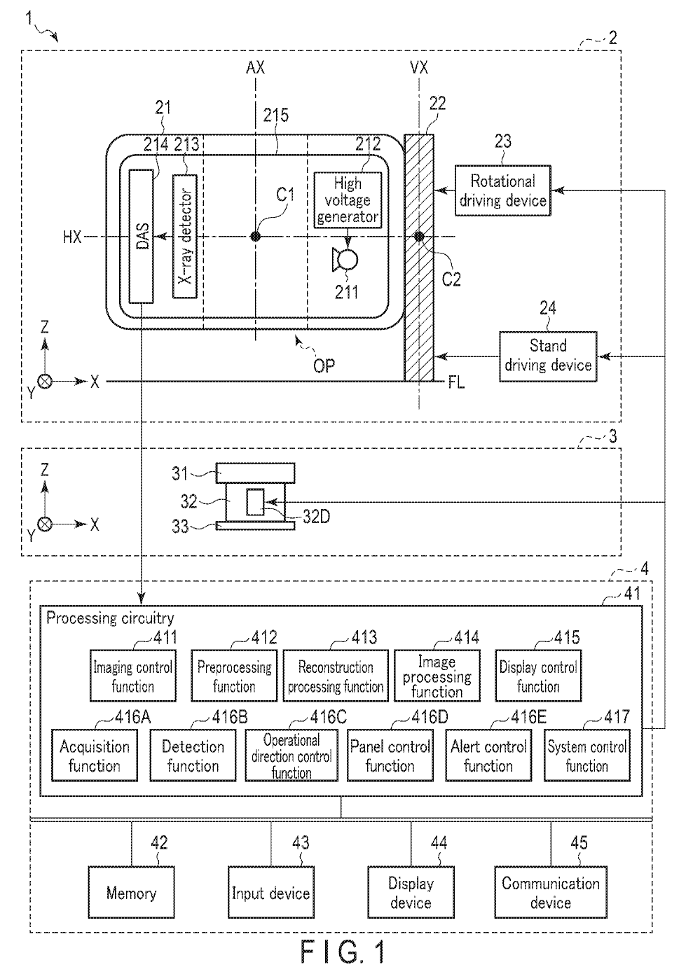

FIG. 1 is a diagram illustrating a configuration of an X-ray CT apparatus 1 according to an embodiment. The X-ray CT apparatus 1 is an apparatus for X-ray CT imaging. The X- ray CT apparatus 1 includes a gantry 2, a bed 3 and a console 4. For example, the gantry 2 and the bed 3 are installed in an examination room, and the console 4 is installed in an operation room that neighbors the examination room. The gantry 2, bed 3 and console 4 are mutually communicably connected by wire or wirelessly.

### Configuration of Gantry

The gantry 2 is an apparatus that executes X-ray CT imaging. The gantry 2 includes a scanner 21, a stand 22, a rotational driving device 23 and a stand driving device 24.

In regard to the gantry 2, a three-dimensional orthogonal coordinate system is defined. The orthogonal coordinate system includes an X axis, a Y axis and a Z axis, which are perpendicular to each other. An X axis direction is a direction (also referred to as "first horizontal direction") that is parallel to a floor surface FL of the examination room and passes through the stand 22 from a center C1 of an opening OP of the scanner 21. A Y axis direction is a direction (also referred to as "second horizontal direction") that is parallel to the floor surface FL of the examination room and is perpendicular to the X axis. A Z axis direction is a direction (also referred to as "vertical direction") that is perpendicular to the X axis and the Y axis.

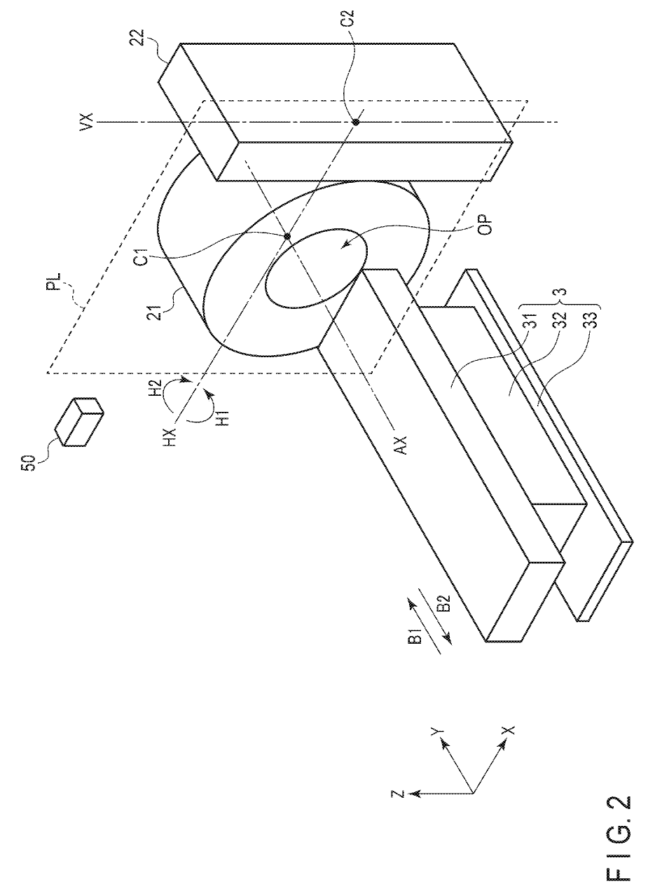

The scanner 21 is an apparatus that accommodates an imaging mechanism for executing X-ray CT imaging. The scanner 21 includes the opening OP (also referred to as "bore") of a cylindrical shape, and the imaging mechanism provided around a center axis AX of the opening OP. The shape of the scanner 21 may be a cylindrical shape or a prismatic shape (in particular, a quadratic prismatic shape) including the opening OP. The scanner 21 includes, as the imaging mechanism, an X-ray tube 211, a high voltage generator 212, an X-ray detector 213, a DAS 214, and a rotating frame 215 (see also FIGS. 2, 3 and 4).

The center axis AX of the scanner 21 is an axis corresponding to a center line of the opening OP. In standup imaging (i.e., in a state illustrated in FIG. 1), the center axis AX is perpendicular to the floor surface FL of the examination room. A horizontal axis HX of the scanner 21 is an axis extending in a horizontal direction (in particular, the X axis direction) perpendicular to the center axis AX. The horizontal axis HX is also referred to as "tilt axis". At a time when the scanner 21 rotates around the horizontal axis HX, the center axis AX also rotates in the same direction. An intersection between the center axis AX and the horizontal axis HX is the center C1 of the opening OP.

Note that the rotating or swinging of the scanner 21 around the horizontal axis HX may also be referred to as "tilting". Hereinafter, a state in which the center axis AX of the opening OP is substantially parallel to the vertical direction is referred to as "non-tilt state". A state in which the center axis AX of the opening OP is inclined with respect to the vertical direction is referred to as "tilt state". An inclination angle of the center axis AX of the opening OP with respect to the vertical direction is referred to as "tilt angle". The range of the tilt angle is, for example, ±90 degrees, with the non-tilt state being set as 0 degrees. The tilt state may be set by tilting the scanner 21 around a rotational axis that is different from the horizontal axis HX, or may be set by a part of the scanner 21 being moved in an up-and-down direction.

The X-ray tube 211 radiates X-rays on a subject (not illustrated). The X-ray tube 211 is connected to the high voltage generator 212 via a high-voltage cable. The X-ray tube 211 includes a cathode that generates thermoelectrons, an anode that receives the thermoelectrons flying from the cathode and generates X-rays, and a vacuum tube that holds the cathode and the anode. A tube voltage is applied by the high voltage generator 212 between the cathode and the anode. By the application of the tube voltage, thermoelectrons fly from the cathode toward the anode. By the thermoelectrons flying from the cathode toward the anode, the tube current flows. By the thermoelectrons impinging upon the anode, X-rays are generated.

The high voltage generator 212 includes electric circuitry such as a transformer and a rectifier. The high voltage generator 212 generates a high voltage that is applied to the X-ray tube 211, and a filament current that is supplied to the X-ray tube 211. The high voltage generator 212 may be of a transformer type or of an inverter type. The high voltage generator 212 may be provided on the rotating frame 215, or may be provided on a fixing frame (to be described later) that holds the rotating frame 215.

The X-ray detector 213 detects X-rays that are radiated from the X-ray tube 211 and pass through the subject, and outputs an electric signal corresponding to the dose of detected X-rays to the DAS 214. The X-ray detector 213 has a structure (one-dimensional structure) in which a plurality of X-ray detection elements are arrayed in one dimension in a channel direction. Alternatively, the X-ray detector 213 has a structure (two-dimensional structure) in which a plurality of X-ray detection elements are arrayed in two dimensions in a channel direction and a slice direction.

The X-ray detector 213 is, for example, of an indirect conversion type in which incident X-rays are converted into light and the light is converted into an electric signal. The X-ray detector 213 of the indirect conversion type includes a grid, a scintillator array, and an optical sensor array. The grid includes an X-ray shield plate that is disposed on the side of an X-ray incidence surface of the scintillator and absorbs scattered X-rays. The grid is also referred to as "collimator" (one-dimensional collimator or two-dimensional collimator). The scintillator array includes a plurality of scintillators. The scintillator outputs light of a light amount corresponding to an incident X-ray dose. The optical sensor array converts the light from the scintillator into an electric signal corresponding to the light amount. As the optical sensor, for example, a photodiode is used.

Note that the X-ray detector 213 may be of a direct conversion type that converts incident X-rays into an electric signal. The X-ray detector 213 may be of a photon counting type that counts photons of incident X-rays for each of energy bins.

The DAS 214 reads, from the X-ray detector 213, an electric signal corresponding to the dose of X-rays detected by the X-ray detector 213. The DAS 214 amplifies the read electric signal and integrates the electric signal during a view period, thereby collecting detection data having digital values corresponding to the dose of X-rays during the view period. The detection data is also called "projection data". The DAS 214 is implemented by an application specific integrated circuit (ASIC) in which a circuit element capable of generating projection data is mounted. The projection data is transmitted to the console 4 via a non-contact data transmission device or the like.

The rotating frame 215 is an annular frame that rotatably supports the X-ray tube 211 and the X-ray detector 213 around the center axis AX. Specifically, the rotating frame 215 supports the X-ray tube 211 and the X-ray detector 213 in such a manner that the X-ray tube 211 and the X-ray detector 213 are opposed to each other. The rotating frame 215 further supports the high voltage generator 212 and the DAS 214, in addition to the X-ray tube 211 and the X-ray detector 213. The rotating frame 215 is supported by a fixing frame in such a manner as to be rotatable around the center axis AX. By the rotating frame 215 rotating around the center axis AX, the X-ray tube 211 and the X-ray detector 213 rotate around the center axis AX. The rotating frame 215 is an example of a rotating unit.

The stand 22 is a structure that supports a side surface of the scanner 21. The stand 22 is installed on the floor surface FL of the examination room. The stand 22 rotatably supports the scanner 21 such that the scanner 21 is rotatable around the horizontal axis HX. The stand 22 movably supports the scanner 21 such that the scanner 21 is movable along the vertical direction. The stand 22 includes a rotating mechanism (not illustrated) for rotating (or tilting) the scanner 21 around the horizontal axis HX. The stand 22 includes a moving mechanism (not illustrated) for moving (or sliding) the scanner 21 in the vertical direction (see also FIGS. 2, 3 and 4).

The stand 22 has a symmetric shape with respect to a vertical axis VX passing through a center C2 of the stand 22. A positional relationship between units of the stand 22 is, for example, symmetric with respect to the vertical axis VX. An intersection between the vertical axis VX and the horizontal axis HX is the center C2 of the stand 22. The stand 22 has, for example, a prismatic shape or a cylindrical shape. The stand 22 may have any shape if the stand 22 has a shape that is symmetric around the vertical axis VX. For example, the stand 22 may have a curved surface. It is preferable that the stand 22 in a state in which a cover is attached to the stand 22 has a shape that is symmetric around the vertical axis VX.

The rotational driving device 23 is a device that drives the rotation of the rotating frame 215. The rotational driving device 23 includes a motor (for example, direct drive motor, servo motor). The rotational driving device 23 drives the motor and generates driving force in accordance with the control by the console 4. The rotational driving device 23 supplies the generated driving force to the rotating frame 215.

The stand driving device 24 is a device that drives various mechanisms included in the stand 22. The stand driving device 24 includes a motor (for example, direct drive motor, servo motor). The stand driving device 24 drives the motor and generates driving force in accordance with the control by the console 4. The stand driving device 24 supplies the generated driving force to the rotating mechanism (described above) of the stand 22. The stand driving device 24 supplies the generated driving force to the moving mechanism (described above) of the stand 22.

### Configuration of Bed

The bed 3 is an apparatus that places a subject thereon, and moves the subject. In regard to the bed 3, like the gantry 2, a three-dimensional orthogonal coordinate system is defined. The bed 3 is installed on one side of the opening OP of the scanner 21. The bed 3 includes a top 31, a base 32 and a bottom plate 33 (see also FIG. 2).

The top 31 is a plate on which the subject is placed. The top 31 is configured to be movable in a freely selected axis direction (i.e., X axis direction, Y axis direction, Z axis direction) via a support frame (not illustrated). A minor axis direction of the top 31 corresponds to the X axis direction. A major axis direction of the top 31 corresponds to the Y axis direction.

The base 32 is a housing that supports the top 31. The base 32 includes a top driving device 32D. The top driving device 32D includes a motor (for example, direct drive motor, servo motor). The top driving device 32D drives the motor and generates driving force in accordance with the control by the console 4. The top driving device 32D supplies the generated driving force to the support frame (described above).

The bottom plate 33 is a plate that supports the base 32, and is installed on the floor surface FL of the examination room.

FIG. 2 is a perspective view illustrating a configuration of the scanner 21, the stand 22 and the bed 3. In supine position imaging (i.e., a state illustrated in FIG. 2), the center axis AX is parallel to the floor surface FL of the examination room (in particular, the Y axis direction). As a result, the center axis AX in the "supine position imaging" corresponds to an axis acquired by rotating the center axis AX in the "standup imaging" over 90 degrees around the horizontal axis HX.

The bed 3 is installed on one side of the opening OP of the scanner 21. In the example of FIG. 2, the stand 22 is disposed on the right side as viewed from the bed 3 (i.e., the right-hand cantilever structure). As regards the scanner 21 and the stand 22, one side on which the bed 3 is installed is referred to as "forward side" (or "front side"), and the other side on which the bed 3 is not installed is referred to as "backward side" (or "rear side"). A direction passing through the "forward side" and "backward side" (i.e., the Y axis direction) is also referred as "front-and-back direction").

The scanner 21 can rotate in an anticlockwise direction H1, in a case where the horizontal axis HX is viewed from the stand 22. The scanner 21 can rotate in an clockwise direction H2, in a case where the horizontal axis HX is viewed from the stand 22. The scanner 21 can rotate in the direction H1 or the direction H2 in accordance with the control by the console 4.

Preferably, the scanner 21 has a symmetric shape with respect to a plane PL that passes through the center C1 of the opening OP and is perpendicular to the center axis AX, but may have an asymmetric shape. The plane PL may coincide with, for example, at least one of planes (i.e., imaging planes) that are formed by an X-ray beam radiated from the X-ray tube 211 onto the X-ray detector 213. It is preferable that the plane PL coincides with a plane positioned at the center in the direction of the center axis AX, among the planes formed by the X-ray beam.

The scanner 21 may include, with respect to the plane PL, at least one of a code, a pattern and a device for electrically identifying one side of the plane PL. The scanner 21 may include the code or the like on a part on the one side of the scanner 21, and may not include the code or the like on a part on the other side. The code is, for example, a QR (Quick Response) code (trademark). The pattern may be, for example, a pattern of visible light, or a pattern by a paint reacting to infrared, ultraviolet or the like. The device may be a signal transmitter or a position sensor. As a result, the console 4 can identify the front side and the rear side of the scanner 21, based on the code or the like.

Note that a camera 50 (to be described later) may electrically identify one side of the plane PL. For example, the camera 50 photographs the scanner 21 and the bed 3. Based on the photographed image, the camera 50 may identify a side on which the bed 3 is disposed in relation to the scanner 21, and a side on which the bed 3 is not disposed in relation to the scanner 21.

The scanner 21 may include, with respect to the plane PL, a design, a pattern and a color for a user to visually identify one side of the plane PL. The scanner 21 may include the design or the like on a part on the one side of the scanner 21, and may not include the design or the like on a part on the other side of the scanner 21. The design may be a recess, a projection, or an opening. The pattern may be a pattern of visible light, or may be a pattern of a paint reacting to infrared, ultraviolet or the like. The color may be red, green, blue, or the like. As a result, the user can identify the front side and the rear side of the scanner 21, based on the design or the like.

Similarly, the stand 22 may include, with respect to a plane (for example, plane PL) that passes through the center C2 of the stand 22 and is parallel to the vertical axis VX and that passes through a side surface of the scanner 21, at least one of a code, a pattern and a device for electrically identifying one side of the plane. The stand 22 may include the code or the like on a part on the one side of the stand 22. As a result, the console 4 can identify the front side and the rear side of the stand 22, based on the code or the like.

Similarly, the stand 22 may include, with respect to a plane (for example, plane PL) that passes through the center C2 of the stand 22 and is parallel to the vertical axis VX and that passes through a side surface of the scanner 21, at least one of a design, a pattern and a color for the user to visually identify one side of the plane. The stand 22 may include the design or the like on a part on the one side of the stand 22. As a result, the user can identify the front side and the rear side of the stand 22, based on the design or the like.

Note that in a case where the scanner 21 rotates around the horizontal axis HX, the plane PL similarly rotates around the horizontal axis HX. With reference to the plane (described above) different from the plane PL, the stand 22 may include a structure for electrically or visually identify one side of the plane. As a result, the console 4 or the user can recognize the front side or the rear side of the stand 22, regardless of the rotation of the scanner 21 around the horizontal axis HX.

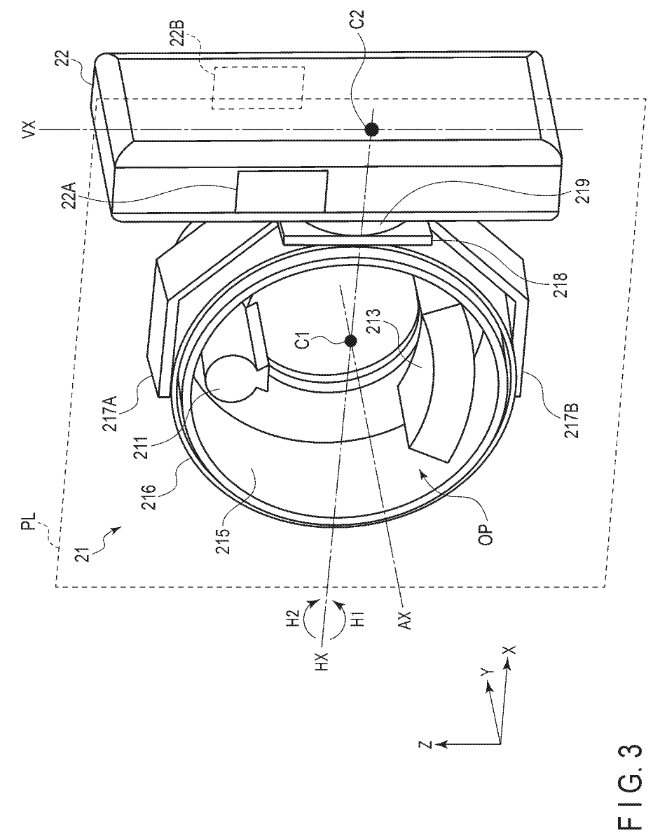

Note that the rotating frame 215 (see FIG. 3) includes a first surface (side surface) of a cylindrical shape, and a second surface (bottom surface) of an annular shape, which is connected to the first surface. The second surface is located on a rear side (i.e., a positive direction side of the Y axis) of the X-ray tube 211 and the X-ray detector 213. On the other hand, a surface corresponding to the second surface is not present on a front side (i.e., a negative direction side of the Y axis) of the X-ray tube 211 and the X-ray detector 213. Specifically, the rotating frame 215 has an asymmetric shape with respect to the plane PL.

The scanner 21 may include at least one of a code, a pattern and a device, which indicate information relating to the position of the second surface in relation to the plane PL. Alternatively, the scanner 21 may include at least one of a code, a pattern and a device, which indicate information relating to the position of the opening of the rotating frame 215, which is located on an opposite side to the second surface with respect to the plane PL. The scanner 21 may include at least one of a design, a pattern and a color, which visually indicate information relating to the position of the second surface in relation to the plane PL. Alternatively, the scanner 21 may include at least one of a design, a pattern and a color, which visually indicate information relating to the position of the opening of the rotating frame 215, which is located on the opposite side to the second surface with respect to the plane PL.

The bed 3 can move (slide) the top 31 toward the inside of the opening OP of the scanner 21. The top 31 can move in a Y axis direction B1 toward the inside of the opening OP. The top 31 can move in a Y axis direction B2 away from the inside of the opening OP. The top 31 can move in the direction B1 or the direction B2 in accordance with the control by the console 4.

The camera 50 is disposed above the scanner 21, stand 22 and bed 3 (for example, on a ceiling surface of the examination room). The camera 50 is disposed at such a position as to be capable of photographing at least the scanner 21 and the bed 3. The camera 50 photographs at least the scanner 21 and the bed 3 in accordance with the control by the console 4. The camera 50 may photograph the stand 22. The camera 50 transmits a photographed image to the console 4.

FIG. 3 is a perspective view illustrating a detailed configuration of the scanner 21 and the stand 22. FIG. 3 illustrates a front side of the scanner 21 in a state in which a cover covering the surface of the scanner 21 is removed. For the convenience of description, the high voltage generator 212 and the DAS 214 are not illustrated. In the scanner 21, the X-ray tube 211 and the X-ray detector 213 are mounted on the rotating frame 215. The scanner 21 includes a fixing frame 216, a first arm 217A, a second arm 217B, a fixing plate 218 and a bearing 219.

The fixing frame 216 is an annular frame that rotatably supports the rotating frame 216 around the center axis AX. The fixing frame 216 may support the rotating frame 215 through a slip ring and a brush (not illustrated). The fixing frame 216 is an example of a fixing unit.

The first arm 217A and the second arm 217B are members that support one end portion and the other end portion of the fixing frame 216. The first arm 217A and second arm 217B clamp the fixing frame 216 from both end portions. The first arm 217A and second arm 217B may clamp the fixing frame 216 in such a manner as to surround the entirety of the side surface of the fixing frame 216. Each of the first arm 217A and second arm 217B is an example of the fixing unit.

The fixing plate 218 is a member that fixes the first arm 217A and second arm 217B to the bearing 219. The fixing plate 218 has, for example, a plate shape. The fixing plate 218, together with the first arm 217A and second arm 217B, may be formed as a single unit. The fixing plate 218 is an example of the fixing unit.

The bearing 219 is a member that rotatably supports the fixing plate 218 around the horizontal axis HX. The bearing 219 may rotate the fixing plate 218 around the horizontal axis HX by receiving driving force from a motor (for example, stand driving device 24). The bearing 219 is an example of the fixing unit.

The various units (for example, fixing frame 216, first arm 217A, second arm 217B, fixing plate 218 and bearing 219) that fix the rotating frame 215 are, for example, symmetric with respect to the horizontal axis HX. Specifically, the various units have symmetric structures with respect to the horizontal axis HX. In addition, the positional relationship between the various units that fix the rotating frame 215 is symmetric with respect to the horizontal axis HX. The various units may be symmetric (i.e., vertically symmetric) with respect to a horizonal plane (i.e., XY plane). The stand 22 may be symmetric (i.e., symmetric in the front-and-rear direction) with respect to a vertical plane (in particular, XZ plane).

The stand 22 includes a first operation panel 22A on a first side on which the bed 3 is installed, and a second operation panel 22B on a second side opposite to the first side. Specifically, the first operation panel 22A is disposed on the front side of the stand 22, and the second operation panel 22B is disposed on the rear side of the stand 22. The console 4 executes control to switch the ON/OFF of power sources of the first operation panel 22A and the second operation panel 22B. The console 4 may switch, for example, the ON/OFF of a sleep mode in regard to the screen display of the first operation panel 22A and the second operation panel 22B. The first operation panel 22A and the second operation panel 22B may display a control screen or a GUI (Graphical User Interface) for controlling the operation of the X-ray CT apparatus 1. The operation of the X-ray CT apparatus 1 is, for example, an operation relating to an elevation or rotation of the scanner 21, a movement of the bed 3, X-ray irradiation, or the like.

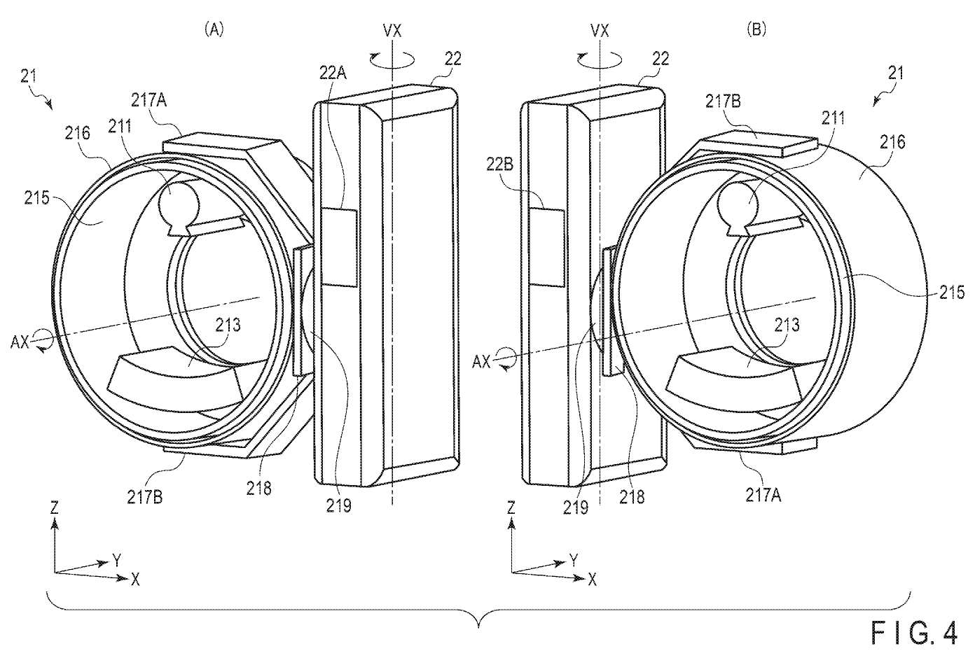

FIG. 4 is a perspective view illustrating a right-hand cantilever structure and a left-hand cantilever structure. In order to change the state of the right-hand cantilever structure (see part (A) of FIG. 4) as viewed from the negative direction of the Y axis to the left-hand cantilever structure (see part (B) of FIG. 4) as viewed from the negative direction of the Y axis, the fixing unit of the scanner 21 needs to be rotated by a half circumference around the center axis AX, and the stand 22 needs to be rotated by a half circumference around the vertical axis VX.

As described above, the fixing unit of the scanner 21 is symmetric with respect to the horizontal axis HX that is perpendicular to the center axis AX, and the stand 22 is symmetric with respect to the vertical axis VX. Accordingly, the fixing unit of the scanner 21 functions even if the fixing unit of the scanner 21 is vertically inverted by being rotated around the center axis AX over 180 degrees, and the stand 22 functions even if the stand 22 is reversed in the front-and-rear direction by being rotated around the vertical axis VX over 180 degrees. Specifically, by using common hardware (or parts), the X-ray CT apparatus 1 can implement the right-hand cantilever structure in which the stand 22 is positioned on the right side as viewed from the bed 3, and can implement the left-hand cantilever structure in which the stand 22 is positioned on the left side as viewed from the bed 3.

Since the fixing frame 216, first arm 217A, second arm 217B, fixing plate 218 and bearing 219 of the scanner 21 are symmetric with respect to the horizontal axis HX, the height of the scanner 21 at a time of supine position imaging can be commonly set between the right-hand cantilever structure and the left-hand cantilever structure. In addition, in each of the right-hand cantilever structure and the left-hand cantilever structure, the height of the top 31 can commonly be set in a predetermined workflow, such as positioning before supine position imaging. Moreover, in a case where the plane PL coincides with a plane located at the center in the direction of the center axis AX among a plurality of planes formed by the X-ray beam, the planes formed by the X-ray beam similarly spread with respect to the plane PL in each of the right-hand cantilever structure and the left-hand cantilever structure. Thus, the distance and layout between the scanner 21 and stand 22, and the bed 3, can commonly be set between the right-hand cantilever structure and the left-hand cantilever structure. Accordingly, the difference in feeling of use by the user between the right-hand cantilever structure and the left-hand cantilever structure can be decreased.

### Configuration of Console

The description returns to FIG. 1. The console 4 is a computer that controls the gantry 2 and the bed 3. The console 4 controls the high voltage generator 212, DAS 214, rotational driving device 23 and stand driving device 24, which are included in the gantry 2. The console 4 controls the top driving device 32D included in the bed 3. The console 4 may control the camera 50, the first operation panel 22A, and the second operation panel 22B.

The console 4 includes, as various structural elements, processing circuitry 41, a memory 42, an input device 43, a display device 44, and a communication device 45. The various structural elements are mutually communicably coupled via a bus (BUS). At least some of the various structural elements may be included in the gantry 2 or the bed 3.

The processing circuitry 41 is circuitry that comprehensively controls the entire operation of the console 4. The processing circuitry 41 includes at least one processor. The processor is a CPU (Central Processing Unit), a GPU (Graphics Processing Unit), an ASIC (Application Specific Integrated Circuit), a PLD (Programmable Logic Device), or the like. The PLD is an SPLD (Simple Programmable Logic Device), a CPLD (Complex Programmable Logic Device), an FPGA (Field Programmable Gate Array), or the like. The processing circuitry 41 is an example of a processing unit.

In a case where the processor is a CPU, the CPU implements various functions by reading and executing various programs stored in the memory 42. In a case where the processor is an ASIC, various functions are embedded in the inside of the ASIC as logic circuitries. The processor may be constructed as single circuitry, or may be constructed as a combination of circuitries. The processor implements, as various functions, an imaging control function 411, a preprocessing function 412, a reconstruction processing function 413, an image processing function 414, a display control function 415, an acquisition function 416A, a detection function 416B, an operational direction control function 416C, a panel control function 416D, an alert control function 416E, and a system control function 417.

The imaging control function 411 is a function of controlling X-ray CT imaging by the gantry 2. The imaging control function 411 controls the high voltage generator 212, DAS 214, rotational driving device 23 and stand driving device 24, in such a manner that the gantry 2 executes X-ray CT imaging in accordance with a predetermined imaging condition. The imaging control function 411 stores projection data received from the DAS 214 in the memory 42. The imaging control function 411 is an example of an imaging control unit.

The preprocessing function 412 is a function of executing preprocessing on the projection data. The preprocessing function 412 executes various preprocessing (for example, logarithmic conversion, offset correction, sensitivity correction, beam hardening correction) on the projection data in accordance with a predetermined preprocessing condition, thereby generating projection data after preprocessing. The preprocessing function 412 stores the projection data after preprocessing in the memory 42. The preprocessing function 412 is an example of a preprocessing unit.

The reconstruction processing function 413 is a function of executing reconstruction processing on the projection data after preprocessing. The reconstruction processing function 413 executes various reconstruction processing (for example, filter back projection, iterative reconstruction) on the preprocessed projection data in accordance with a predetermined reconstruction processing condition, thereby generating reconstructed image data (i.e., volume data). The reconstruction processing function 413 stores the reconstructed image data in the memory 42. The reconstruction processing function 413 is an example of a reconstruction processing unit.

The image processing function 414 is a function of executing image processing on the reconstructed image data. The image processing function 414 executes various image processing (for example, multi-planar reconstruction (MPR), maximum intensity projection (MIP), volume rendering) on the reconstructed image data in accordance with a predetermined image processing condition, thereby generating CT image data. The image processing function 414 stores the CT image data in the memory 42. The image processing function 414 is an example of an image processing unit.

The display control function 415 is a function of controlling display of a CT image based on the CT image data. The display control function 415 executes window processing on the CT image data in such a manner that the display device 44 displays the CT image in accordance with a predetermined display condition. The display control function 415 transmits the CT image data after the window processing to the display device 44. The display control function 415 may display various images on the first operation panel 22A and the second operation panel 22B. The display control function 415 is an example of a display control unit.

The acquisition function 416A is a function of acquiring various data. The acquisition function 416A acquires a correct positional relationship CP between the scanner 21 and the bed 3. The correct positional relationship CP may be a relative positional relationship between coordinates (X, Y, Z) of the scanner 21 and the bed 3 in the three-dimensional orthogonal coordinate system, or may be an absolute positional relationship between the scanner 21 and the bed 3 with reference to the origin. The coordinates of the scanner 21 are, for example, coordinates of the center C1 of the scanner 21. The coordinates of the bed 3 are, for example, coordinates of the center of the bed 3. The acquisition function 416A is an example of an acquisition unit.

The correct positional relationship CP is stored in the memory 42 at a manufacturing stage of the X-ray CT apparatus 1, for example, as the information of positions where the scanner 21, the stand 22 and the bed 3 of the X-ray CT apparatus 1 are installed in the examination room. The correct positional relationship CP may include installation information indicating whether the X-ray CT apparatus 1 is installed in the examination room with the right-hand cantilever structure or with the left-hand cantilever structure. In this case, the installation information, and correlation information in which information pieces of positions where the scanner 21, stand 22 and bed 3 are installed in the examination room, are stored in the memory 42, and the correct positional relationship CP can be obtained by referring to the installation information and the correlation information.

The detection function 416B is a function of detecting various data. For example, based on an image photographed by the camera 50, the detection function 416B detects an actual positional relationship AP between the scanner 21 and the bed 3. The actual positional relationship AP may be a relative positional relationship between coordinates (X, Y, Z) of the scanner 21 and the bed 3 in the three-dimensional orthogonal coordinate system, or may be an absolute positional relationship between the scanner 21 and the bed 3 with reference to the origin. The detection function 416B is an example of a detection unit.

The operational direction control function 416C is a function of controlling directions of various operations. Based on the actual positional relationship AP between the scanner 21 and the bed 3, the operational direction control function 416C controls the direction of the operation of at least one of the scanner 21 and the bed 3. Responding to an input via an identical input interface, the operational direction control function 416C switches the direction (i.e., direction H1 or direction H2) of the operation of rotating the scanner 21 around the horizontal axis HX perpendicular to the center axis AX, based on the actual positional relationship AP. For example, the operational direction control function 416C switches a signal of the input interface received from the user, which indicates the rotational operation in the direction H1 in the case of the right-hand cantilever structure, to a signal indicating the rotational operation in the direction H2 in the case of the left-hand cantilever structure. In this manner, the rotational direction in which an upper part of the scanner 21 inclines toward the bed 3 can be implemented by the identical input interface in the case of each of the right-hand cantilever structure and the left-hand cantilever structure. Specifically, since the user can perform an operation via the same input interface, the difference in feeling of use by the user between the right-hand cantilever structure and the left-hand cantilever structure decreases.

The operational direction control function 416C switches the direction of moving the bed 3, in particular, the top 31, based on the actual positional relationship AP. For example, the operational direction control function 416C switches a signal of the input interface received from the user, which indicates the movement of the top 31 in the positive direction of the Y axis in the case of the right-hand cantilever structure, to a signal indicating the movement of the top 31 in the positive direction of the Y axis in the case of the left-hand cantilever structure. In this manner, the operation in the direction in which the top 31 approaches the scanner 21 can be implemented by the identical input interface in the case of each of the right-hand cantilever structure and the left-hand cantilever structure. Specifically, even in the case of either the right-hand cantilever structure or the left-hand cantilever structure, the same input interface, for example, the same layout of buttons, can be adopted. The operational direction control function 416C is an example of an operational direction control unit.

Note that the operational direction control function 416C may control the initial position (or home position) of the X-ray tube 211. In a case where the conversion between the right-hand cantilever structure and the left-hand cantilever structure is executed, the rotating frame 215 on which the X-ray tube 211 is mounted rotates over 180 degrees around the center axis AX together with the fixing frame 216. After this rotation, the position of the X-ray tube 211 is inverted with respect to the center axis AX. Then, the operational direction control function 416C may keep the initial position of the X-ray tube 211 at the same position by rotating once again the rotating frame 215 over 180 degrees around the center axis AX.

The panel control function 416D is a function of controlling various panels. Based on the actual positional relationship AP between the scanner 21 and the bed 3, the panel control function 416D switches the first operation panel 22A to the ON state, and switches the second operation panel 22B to the OFF state. For example, in the case of the right-hand cantilever structure, the panel control function 416D switches the first operation panel 22A to the ON state and switches the second operation panel 22B to the OFF state, and thereby only the operation panel located on the side of the bed 3 can be set in the ON state. The panel control function 416D is an example of a panel control unit.

The alert control function 416E is a function of controlling various alerts. For example, in a case where the correct positional relationship CP between the scanner 21 and the bed 3 does not coincide with the actual positional relationship AP, the alert control function 416E causes an alarm (not illustrated) to issue an alert. For example, in the case where the correct positional relationship CP between the scanner 21 and the bed 3 does not coincide with the actual positional relationship AP, the alert control function 416E causes the operation panel to display text such as "Confirm installation positions of the scanner and the bed". The alert control function 416E is an example of an alert control unit.

The system control function 417 is a function of comprehensively controlling the entire operation of the processing circuitry 41. The system control function 417 may control the various functions included in the processing circuitry 41, based on various input operations received from the user via the input device 43. The system control function 417 is an example of a system control unit.

The memory 42 is a device that stores various data. The memory 42 is a storage device such as an HDD (Hard Disk Drive), an SSD (Solid State Drive), a RAM (Random Access Memory), or a ROM (Read Only Memory). The memory 42 may be a storage medium such as a CD (Compact Disc), a DVD (Digital Versatile Disc), or a flash memory. The memory 42 stores projection data, reconstruction image data, CT image data, and the like. The memory 42 is an example of a storage unit.

The input device 43 is a device that receives various input operations from the user. The input device 43 is a mouse, a keyboard, a trackball, a switch, a button, a joystick, a touch pad, a tablet terminal, or the like. The input device 43 converts a received input operation into an electric signal, and transmits the converted electric signal to the processing circuitry 41. The input device 43 receives various conditions (for example, imaging condition, preprocessing condition, reconstruction processing condition, image processing condition, display condition) from the user. The input device 43 is an example of an input unit.

The display device 44 is a device that displays various images. The display device 44 is an LCD (Liquid Crystal Display), an OELD (Organic Electro Luminescence Display), or the like. The display device 44 displays a CT image based on CT image data. The display device 44 may display a GUI for receiving various input operations from the user. The display device 44 is an example of a display unit.

The communication device 45 is a device for communicating various data. The communication device 45 communicates CT image data, based on a DICOM (Digital Imaging Communication in Medicine) standard. The communication device 45 may communicate CT image data with an external device that is connected to the console 4 via a network. The communication device 45 is an example of a communication unit.

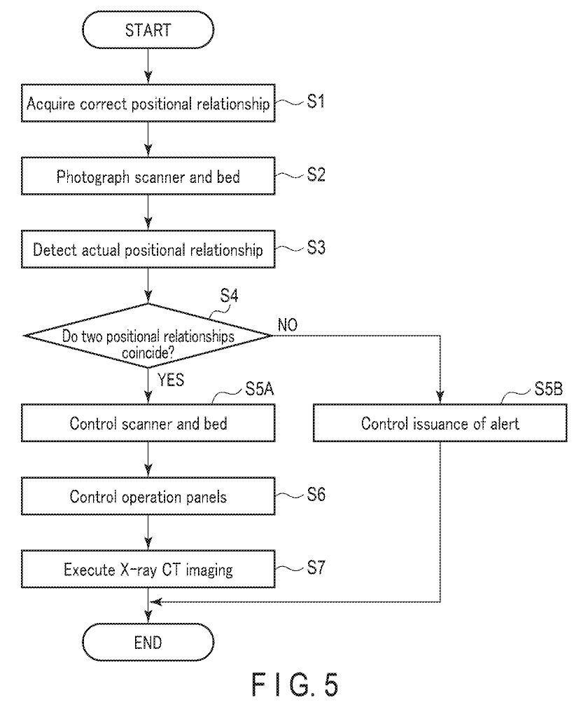

FIG. 5 is a flowchart illustrating an operation of the X-ray CT apparatus 1 according to the embodiment. The X-ray CT apparatus 1 executes steps S1 to S7 through the various functions included in the processing circuitry 41.

### Step S1

The acquisition function 416A acquires the correct positional relationship CP. Specifically, the acquisition function 416A acquires the correct positional relationship CP from the memory 42. The acquisition function 416A may acquire the correct positional relationship CP, based on an operation that is input by the user through the input device 43.

### Step S2

The camera 50 photographs the scanner 21 and the bed 3. Specifically, the acquisition function 416A causes the camera 50 to photograph the scanner 21 and the bed 3. The acquisition function 416A acquires an image photographed by the camera 50 through the communication device 45. Step S2 may be executed before step S1.

### Step S3

The detection function 416B detects the actual positional relation AP. Specifically, the detection function 416B detects the actual positional relation AP between the scanner 21 and the bed 3, based on the image photographed in step S2. The detection function 416B may detect the actual positional relationship AP, by using a machine learning model or the like that is trained for detecting the position of an object.

### Step S4

The alert control function 416E determines whether the two positional relationships coincide or not. Specifically, the alert control function 416E determines whether the correct positional relationship CP acquired in step S1 coincides with the actual positional relationship AP detected in step S3. If the two positional relationships coincide (step S4-YES), the process advances to step S5A. If the two positional relationships do not coincide (step S4-NO), the process advances to step S5B.

### Step S5A

The operational direction control function 416C controls the scanner 21 and the bed 3. Specifically, the operational direction control function 416C controls the directions of the operations of the scanner 21 and the bed 3, based on the actual positional relationship AP detected in step S3. First, the operational direction control function 416C switches the direction (i.e., direction H1 or direction H2) in which the scanner 21 is rotated around the horizontal axis HX. Secondly, the operational direction control function 416C switches the direction (i.e., direction B1 or direction B2) in which the bed 3 is moved into the opening OP.

First, in a case where the bed 3 is disposed on the front side of the scanner 21 (see FIG. 2), the operational direction control function 416C switches the direction H1 to a position direction (+) and switches the direction H2 to a negative direction (-). Conversely, in a case where the bed 3 is disposed on the rear side of the scanner 21, the operational direction control function 416C switches the direction H1 to a negative direction (-) and switches the direction H2 to a positive direction (+). As a result, since the operational direction control function 416C keeps identical the directivity relating to the rotation around the horizontal axis HX of the scanner 21 in relation to the bed 3, the user can intuitively operate the X-ray CT apparatus 1.

Secondly, in a case where the bed 3 is disposed on the front side of the scanner 21 (see FIG. 2), the operational direction control function 416C switches the direction B1 to a position direction (+) and switches the direction B2 to a negative direction (-). Conversely, in a case where the bed 3 is disposed on the rear side of the scanner 21, the operational direction control function 416C switches the direction B1 to a negative direction (-) and switches the direction B2 to a positive direction (+). As a result, since the operational direction control function 416C keeps identical the directivity relating to the movement of the bed 3 in relation to the scanner 21, the user can intuitively operate the X-ray CT apparatus 1.

Note that the scanner 21 can be symmetric with respect to the plane PL (i.e., in the front-and-rear direction). In this case, the operational direction control function 416C may not switch the directivity relating to the rotation around the horizontal axis HX of the scanner 21 in relation to the bed 3. Similarly, the operational direction control function 416C may not switch the directivity relating to the movement of the bed 3 in relation to the scanner 21.

### Step S5B

The alert control function 416E controls the issuance of the alert. Specifically, the alert control function 416E causes the alarm to issue the alert, if the correct positional relationship CP acquired in step S1 does not coincide with the actual positional relationship AP detected in step S3. The alert may be sound, light, or the like. As a result, the alert control function 416E can notify the user that the two positional relationships do not coincide. After step S5B, the process ends.

### Step S6

The panel control function 416D controls the operation panels. Specifically, based on the actual positional relationship AP detected in step S3, the panel control function 416D switches the ON/OFF of the first operation panel 22A and the second operation panel 22B. Step S6 may be executed before step S5A.

For example, in a case where the bed 3 is disposed on the front side of the scanner 21 (see FIG. 2 and FIG. 3), the panel control function 416D switches the first operation panel 22A on the front side to the "ON" state, and switches the second operation panel 22B on the rear side to the "OFF" state. Conversely, in a case where the bed 3 is disposed on the rear side of the scanner 21, the panel control function 416D switches the first operation panel 22A on the front side to the "OFF" state, and switches the second operation panel 22B on the rear side to the "ON" state. As a result, the panel control function 416D can save the power consumed by the two operation panels, compared to the case where the two operation panels are always switched to the "ON" state. Moreover, the user can immediately use the operation panel disposed on the side of the bed 3.

### Step S7

The imaging control function 411 executes X-ray CT imaging. Specifically, in the state in which the control in steps S5A and S6 is executed, the imaging control function 411 controls the imaging mechanism or the like of the X-ray CT apparatus 1. After step S7, the process ends.

According to the above-described embodiment, the X-ray CT apparatus 1 includes the scanner 21 and the stand 22. The scanner 21 includes the rotating unit that holds the imaging mechanism around the center axis AX of the opening OP, and the fixing unit that rotatably holds the rotating unit around the center axis AX and has a symmetric shape with respect to the horizontal axis HX perpendicular to the center axis AX. The stand 22 supports a side surface of the scanner 21, and has a symmetric shape with respect to the vertical axis VX perpendicular to the horizontal axis HX.

According to at least one of the above-described embodiments, the commonalty of parts between the right-hand cantilever structure and the left-hand cantilever structure can be improved, as described with reference to FIG. 4.

Besides, in regard to the cantilever X-ray CT apparatus, depending on the layout (or free space) of the examination room in which the cantilever X-ray CT apparatus is installed, there is a case where one of the right-hand cantilever structure and the left-hand cantilever structure is more convenient than the other, from the standpoint of movement lines of the user and the subject, the working space, and the like. Specifically, it is assumed that, as the cantilever X-ray CT apparatus, there is a demand for both the right-hand cantilever structure and the left-hand cantilever structure. According to at least one of the above-described embodiments, since the commonalty of parts between the right-hand cantilever structure and the left-hand cantilever structure is improved, the difference in feeling of use by the user between the right-hand cantilever structure and the left-hand cantilever structure can be decreased, and the intuitive operation is enabled.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray CT apparatus comprising:
a scanner including a rotating unit configured to rotatably hold an imaging mechanism around a center axis of an opening, and a fixing unit configured to rotatably hold the rotating unit around the center axis, the fixing unit having a symmetric shape with respect to a horizontal axis perpendicular to the center axis; and
a stand configured to support a side surface of the scanner, the stand having a symmetric shape with respect to a vertical axis perpendicular to the horizontal axis.

2. The X-ray CT apparatus of claim 1, wherein the scanner has a symmetric shape with respect to a plane that passes through a center of the opening and is perpendicular to the center axis.

3. The X-ray CT apparatus of claim 2, wherein
the imaging mechanism includes an X-ray tube and an X-ray detector, and
the plane coincides with a plane positioned at a center in a direction of the center axis, among a plurality of planes formed by an X-ray beam radiated from the X-ray tube onto the X-ray detector.

4. The X-ray CT apparatus of claim 1, wherein
the rotating unit includes a first surface of a cylindrical shape, and a second surface of an annular shape, the second surface being connected to the first surface, and
the scanner includes at least one of a code and a pattern that indicate information relating to a position of the second surface in relation to a plane that passes through a center of the opening and is perpendicular to the center axis.

5. The X-ray CT apparatus of claim 1, wherein
the rotating unit includes a first surface of a cylindrical shape, and a second surface of an annular shape, the second surface being connected to the first surface, and
the scanner includes at least one of a design and a color that visually indicate information relating to a position of the second surface in relation to a plane that passes through a center of the opening and is perpendicular to the center axis.

6. The X-ray CT apparatus of claim 1, further comprising a bed on which a subject is placed,
wherein the stand includes at least one of a code and a pattern that indicate a side surface closer to the bed, in relation to a plane that is parallel to the vertical axis and passes through the side surface of the scanner.

7. The X-ray CT apparatus of claim 1, further comprising a bed on which a subject is placed,
wherein the stand includes at least one of a design, a pattern and a color that visually indicate a side surface closer to the bed, in relation to a plane that is parallel to the vertical axis and passes through the side surface of the scanner.

8. The X-ray CT apparatus of claim 1, further comprising:
a bed installed on one side of the opening; and
processing circuitry configured to control a direction of an operation of at least one of the scanner and the bed, based on an actual positional relationship between the scanner and the bed.

9. The X-ray CT apparatus of claim 8, wherein the processing circuitry is configured to switch a direction in which the scanner is rotated around the horizontal axis, based on the actual positional relationship.

10. The X-ray CT apparatus of claim 8, wherein the processing circuitry is configured to switch a direction in which the bed is moved into the opening, based on the actual positional relationship.

11. The X-ray CT apparatus of claim 8, wherein
the stand includes a first operation panel on a first side on which the bed is installed, and a second operation panel on a second side opposite to the first side, and
the processing circuitry is configured to switch the first operation panel to an ON state and to switch the second operation panel to an OFF state, based on the actual positional relationship.

12. The X-ray CT apparatus of claim 8, further comprising a camera configured to photograph the scanner and the bed,
wherein the processing circuitry is configured to detect the actual positional relationship, based on an image photographed by the camera.

13. The X-ray CT apparatus of claim 8, wherein the processing circuitry is configured to:
acquire a correct positional relationship between the scanner and the bed; and
cause an alarm to issue an alert, in a case where the correct positional relationship does not coincide with the actual positional relationship.
